(19) United States
(12) Patent Application Publication
(10) Pub. No.: US 2009/0004679 A1
(43) Pub. Date: Jan. 1, 2009

(54) METHOD FOR DETERMINING PLASMINOGEN ACTIVATOR INHIBITOR

(75) Inventors: Yahia Gawad, Mississauga (CA); Tanya Pekatch, Toronto (CA)

Correspondence Address: MYERS BIGEL SIBLEY & SAJOVEC, PO BOX 37428, RALEIGH, NC, 27627, US

(73) Assignee: CardioGenics Inc.

(21) Appl. No.: 12/138,248

(22) Filed: Jun. 12, 2008

Related U.S. Application Data
(63) Continuation of application No. 10/049,727, filed on Jun. 25, 2002, now abandoned.
(60) Provisional application No. 60/131,339, filed on Apr. 28, 1999.

(51) Int. Cl. G01N 33/543; G01N 33/53

(52) U.S. Cl. 435/7.92; 435/7.1; 436/518

(57) ABSTRACT

A method is provided for determining active plasminogen activator inhibitor—Type 1 (PAI-1) in a biological fluid, the method comprising the steps of (i) providing a sample of a biological fluid; and (ii) measuring the amount of PAI-1/multimeric vitronectin complex in the sample to determine active PAI-1 in the sample. A kit for carrying out the method is also provided.

### PRIORITY STATEMENT

The present application is a continuation application of U.S. application Ser. No. 10/049,727, filed Jun. 25, 2006 (abandoned), which is a 35 U.S.C. § 371 national phase application of International Application No. PCT/CA00/00464, filed Apr. 27, 2000 and which claims priority to U.S. Provisional Application No. 60/131,339 filed Apr. 28, 1999, the disclosures of each of which are incorporated herein by reference in their entireties.

### FIELD OF THE INVENTION

This invention relates to the determination of the level of active plasminogen activator inhibitor Type 1 in samples such as biological fluids.

### BACKGROUND OF THE INVENTION

In order to ensure an adequate blood supply to various organs, the mammalian body is equipped with two systems, a coagulation system and a fibrinolytic system. The coagulation system functions to stop bleeding and protect the mammal from blood loss. The fibrinolytic systems functions primarily to dissolve blood clots. The two systems are normally in equilibrium and the enzymes involved in both systems are under control at multiple levels.

The key enzyme of the fibrinolytic systems is a plasmin, which digests the fibrin threads of a fibrin blood clot. Plasmin is formed when its precursor protein, plasminogen, is activated by a plasminogen activator. Plasminogen activators are typical serine proteases and four different plasminogen activator (PA) systems are recognized; (a) factor XII-dependent system, (b) streptokinase (isolated from Streptococci), (c) tissue plasminogen activator (tPA) and (d) urinary plasminogen activator (urokinase or uPA). In humans, only tPA and uPA have physiological importance, tPA being the main fibrinolytic enzyme in the circulation.

The plasminogen activating activity of tPA and uPA is inhibited by several plasminogen activator inhibitors (PAI). Four types of PAI have been described: (a) endothelial-type inhibitor (called Plasminogen Activator Inhibitor Type 1 or PAI-1); (b) placental inhibitor (called Plasminogen Activator Inhibitor Type 2 or PAI-2); (c) heparin-dependent inhibitor (Plasminogen Activator Inhibitor Type 3 or PAI-3); and (d) the protease nexin (Plasminogen Activator Inhibitor Type 4) (Urden et al., (1987), Thromb. Haemost., v. 57, pp. 29-34; Francis et al., (1988), Am. Heart J., v. 115, pp. 776-780; and Kurnik, (1995), Circulation, v. 91, pp. 1341-1346).

Apart from PAI-2 which plays a role in pregnant women, PAI-1 appears to be the only PAI which is important in humans. It is the primary inhibitor of plasminogen activators in the circulation and is secreted into plasma mainly by endothelial cells and the α granules of the platelets. PAI-1 has a great affinity for its target enzymes and, upon binding, both PAI-1 and the plasminogen activator in the formed complex (PAI-1/tPA or PAI-1/uPA) are inactivated. Upon its release from the endothelial cells into the circulation, tPA is quickly captured by PAI-1 and loses activity (more than 95% of tPA in the blood is bound to PAI-1) (Lijnen et al., (1991), J. Biol. Chem., v. 266, pp. 4041-4044).

Previous studies have described the presence of several conformational and functional forms of PAI-1. More than 95% of the total PAI-1 in circulation in humans is found in the platelets, as latent PAI-1. On platelet activation, the latent PAI-1 undergoes a conformational change and is released into the circulation as active PAI-1. The non-platelet PAI-1 in the circulation exists mainly in two forms: inactive PAI-1 or PAI-1 bound to its target enzymes (about 40% of total non-platelet circulating PAI-1) and active PAI-1 or PAI-1 bound to the plasma protein, vitronectin about 60% of total non-platelet circulating PAI-1) (Wagner et al., (1989), J. Clin. Invest., v. 84, pp. 647-655). Circulating complexes of PAI-1 with its target enzymes are largely PAI-1/tPA, with only a minute amount of PAI-1/uPA complex.

Like PAI-1, vitronectin can exist in several conformational states. Platelet vitronectin is present in both monomeric and multimeric forms, whereas plasma vitronectin is reportedly monomeric (Seiffert (1997), J. Biol. Chem., v. 272, p. 9971). If plasma vitronectin is exposed to denaturing agents, multimeric vitronectin is formed, which has exposed epitopes not present in monomeric vitronectin. It has been shown that active PAI-1 binds to multimeric vitronectin with higher affinity than to monomeric vitronectin and that active PAI-1 isolated from plasma is predominantly complexed with a high molecular weight form of vitronectin (Lawrence et al., (1997), J. Biol. Chem., v. 272, p. 7676). Other studies have, however, reported that both monomeric and multimeric vitronectin bind to PAI-1 and, as noted by Lawrence (supra), the nature of the interaction of PAI-1 and vitronectin remains the subject of considerable debate.

Numerous clinical reports have documented that failure of the endogenous fibrinolytic capacity is attributable to an increase in serum PAI-1 activity. Stringer et al., (1994), Arterioscler. Thromb., v. 14, pp. 1452-1458, reported that PAI-1 is released at high concentration from activated platelets and is retained within the thrombus by binding to fibrin, resulting in inhibition of local tPA-mediated clot-lysis. Furthermore, the administration of monoclonal antibodies that block the inhibitory activity of PAI-1 reduced clot lysis resistance. In patients with coronary artery disease (CAD), Hamsten et al., (1985), N. Eng. J. Med., v. 313, pp. 1557-1563, have documented that in young survivors of acute myocardial infarction (AMI), an elevated plasma level of PAI-1 up to 3 years after the event was correlated to a higher rate of reinfarction. Since this initial report, several other investigators have confirmed these observations.

The plasma active PAI-1 level was also investigated, and reported elevated, during the acute coronary thrombotic events. Furthermore, in patients with AMI, the plasma level of PAI-1 was correlated with the capacity to lyse a coronary thrombus. In patients who fail to have restored coronary blood flow, as evident by coronary angiography (determined by angiography 24 hr-1 week after AMI) or by the development of a Q-wave on the ECG, a high plasma level of PAI-1 was documented (Sakamoto et al., (1992), Am. J. Cardiol., v. 70, pp. 271-276 and Ogava, (1993), Cardiol., v. 41, pp. 201-208). From the several studies reported, it can be concluded that in patients with CAD, a high plasma level of PAI-1 is associated with a high risk for developing acute coronary ischemia and that in those who develop an acute event, a high plasma active PAI-1 level is associated with an ominous outcome.

To further establish the role of a balanced equilibrium state between tPA and PAI-1 activities in native fibrinolysis, several clinical trials have investigated patient outcome in artificially induced endothelial dysfunction. In patients who were subjected to Percutaneous Transluminal Coronary Angioplasty (PTCA), the incidence of acute coronary events in the post-PTCA period was correlated with high plasma levels of active PAI-1 around the time of PTCA according to several reports. The incidence of coronary re-stenosis was also investigated and correlated to the levels of plasma PAI-1 (Hara et al., (1995), Cardiology, v. 86, pp. 407-410 and Sakata, (1996), Am. Heart J., v. 131, pp. 1-6).

A further confirmation of the role of PAI-1 in clot lysis was investigated by artificially inhibiting the activity of PAI-1 by either pharmaceuticals or monoclonal antibodies. Levi et al., (1992), Circulation, v. 85, pp. 305-12, have reported that by inhibiting PAI-1 activity through using monoclonal antibodies (Mab), native tPA could lyse a clot. By using N-acetyltetradecapeptide corresponding to the P₁-P₁₄ aminoacid sequence of the PAI-1 to inactivate active PAI-1 and enhance fibrinolysis. Eitzman et al., (1995), J. Clin. Invest., v. 95, pp. 2416-2420, reported that the activity of circulatory PAI-1 decreased, is although antigen level did not and that native tPA was more effective in dissolving the clot. Ohtani et al., Eur. J. Pharmac., v. 197, pp. 151-156, developed a novel inhibitor of PAI-1, (a butadiene derivative called T-686), that has been shown to inhibit thrombosis in two experimental thrombosis models in rats without affecting bleeding time. Friederich, (1997), Circulation, v. 96, pp. 916-921, showed that neutralization of plasma PAI-1 activity by a low molecular weight inhibitor (XR5118) enhances clot lysis and reduces clot growth in a rabbit thrombosis model.

A number of these studies (for example, Eitzman et al., (1995), J. Clin. Invest., v. 95, pp. 2416-2420) indicate the importance of measuring active PAI-1, which was seen to fluctuate while the total level of PAI-1, as determined by immunoassay, remains stable.

The role of active PAI-1 in clot lysis and its relevance in a number of disease states is well established. The availability of an accurate and reliable method to determine the plasma level of active PAI-1 is therefore of great clinical importance.

Previously described methods for determining the level of circulating active PAI-1 have been of two main types, functional or immunological.

Several direct and indirect functional methods to quantify the fibrinolytic inhibition capacity of biological samples have been described. (Verheijen et al., U.S. Pat. No. 4,563,420; Pussard et al., U.S. Pat. No. 5,472,851; Sasamata et al., U.S. Pat. No. 5,102,787). The most commonly used method, Verheijen et al., (1985), Thromb. Res., v. 39, pp. 281-8, measures inhibition of tPA activity, which is primarily due to PAI-1 activity, through the hydrolysis of either a tPA-specific substrate or a plasmin-specific substrate, plasmin having been produced by the action of tPA upon plasminogen. This hydrolysis results in either a measurable chromogenic change or in the breakdown of a fibrin film resulting in measurable clot lysis.

The European Committee of Fibrinolysis evaluated the various functional methods available for measuring tPA inhibition in a multicentre study and concluded that they have limited accuracy, Gram et al., (1993), Thrombosis and Haemostasis, v. 70, pp. 852-857. The main drawbacks of these methods are the presence of a partitioning step of the plasma eugloblins, the non-standardization of the incubation conditions, and of the form and amount of tPA to be utilized and the indirectness of measurements. Also, some of these methods discount the role of plasmin inhibitor activities in the test samples. Another problem encountered in methods of measuring inhibition of tPA functionally is the fact that the activities of both tPA and PAI-1 are unstable and decrease gradually after sample collection. In blood with high PAI-1 levels, the tPA activity can decrease by 50% in about one minute.

In order to avoid the problems encountered with functional assay methods for measuring active PAI-1, several immunoassay methods have been developed. The simplest assays employ an antibody to PAI-1 in a conventional immunoassay (for example, U.S. Pat. Nos. 5,422,245 and 5,629,160). Methods have also been described for measuring active PAI-1 by a two-step procedure: the sample under investigation is divided into two portions and a saturating amount of tPA is added to one portion. The level of PAI-1/tPA complex is then measured in both portions. The difference in the measured amount of the PAI-1/tPA complex between the two portions represents the amount of free or active PAI-1.

Variations on this method have been described, for example, by Amiral et al., (1988), Thrombosis Research, Supplement VIII, pp. 99-113; Sakata et al., U.S. Pat. No. 5,352,583; Niewenhuizen et al., (1995), Blood Coagul. & Fibrinolysis, v. 6, pp. 520-6, and in U.S. Pat. No. 5,352,583.

Utilising pairs of antibodies specific for different parts of the PAI-1/tPA complex in the above-described two-step procedure did provide a more reliable determination of active PAI-1 than the earlier functional assays. There are, nevertheless, problems with the assay based on measuring total PAI-1/tPA complex before and after adding exogenous tPA. For example, special instrumentation and techniques are required to arrest further in vitro binding of tPA to PAI-1. Sample collection is complicated by the need for acidification to prevent any unintended in vitro interaction between tPA and PAI-1 and problems arise from the non-standardisation of the conditions for tPA/PAI-1 binding and of tPA preparations themselves.

Many of these methods are also time consuming and technically demanding, limiting their value in the clinical laboratory,

Methods have been described for measuring complexes of PAI-1 and vitronectin in platelets. For example, Preissner et al., (1989), Blood, v. 74, pp. 1989-1996 used an immunoassay employing anti-PAI-1 and anti-vitronectin antibodies and found evidence of PAI-1/vitronectin complexes in platelets. In contrast, however, Lang et al., (1996), J. Biol. Chem., v. 271, pp. 2754-2761 and Nordenhem et al., (1997), Scand. J. Clin. Invest., v. 57, p. 453, used a similar assay and did not detect such complexes in platelets, casting doubt on the efficacy of such an assay. Nordenhem et al. also noted that the described method was not applicable to plasma, due to interference by the high level of vitronectin in plasma.

There remains a need for improved methods of determining the level of active PAI-1 in circulation.

### SUMMARY OF THE INVENTION

The present invention provides a new method for measuring the level of active PAI-1 in a biological fluid, such as whole blood, plasma or serum.

The method of the invention determines the level of active PAI-1 in circulation by determining the amount of PAI-1 complexed to multimeric vitronectin.

The present invention provides an improved method for determining active PAI-1. The method is much less cumbersome than methods involving comparison of PAI-1/tPA complex levels with and without addition of exogenous tPA. The present method, which measures active PAI-1 directly, as the stable PAI-1/multimeric vitronectin complex, is also less subject to interference from uncontrolled factors such as inconsistencies and artifacts of tPA binding than previously described methods for determining plasma active PAI-1.

In accordance with one embodiment of the invention, a method for determining active plasminogen activator inhibitor-Type I (PAI-1) in a biological fluid comprises the steps:

(i) providing a sample of a biological fluid; and

(ii) measuring the amount of PAI-1/multimeric vitronectin complex in the sample to determine active PAI-1 in the sample.

The biological fluid to be assayed may be selected from the group consisting of whole blood, plasma, serum, saliva, amniotic fluid, cerebrospinal fluid, tissue extract or urine.

In accordance with a further embodiment, a kit for determining active PAI-1 in a biological fluid comprises:

(a) a first antibody which binds selectively to PAI-1; and

(b) a labelled second antibody which binds selectively to multimeric vitronectin.

### DETAILED DESCRIPTION OF THE INVENTION

The present invention provides a method for determining active PAI-1 in a biological fluid by determining the amount of PAI-1/multimeric vitronectin complex present in the fluid.

Any detection reagent or detection system which detects and determines the circulating PAI-1/multimeric vitronectin complex may be employed.

The term “antibody”, as used herein and if not otherwise specified, includes a polyclonal antibody, a monoclonal antibody, a single chain antibody and antibody fragments such as Fab fragments.

As used herein, an antibody is said to “bind selectively” to a target molecule if the antibody recognises and binds the target molecule but does not substantially recognise and bind other molecules present in a sample containing target molecules.

As used herein, an antibody is said to “bind selectively to multimeric vitronectin” if the antibody recognises and binds multimeric vitronectin but does not substantially recognise and bind other molecules, including monomeric vitronectin, present in a sample.

As used herein, “multimeric vitronectin” means a polymer of monomeric vitronectin that occurs naturally in plasma and contains two to four monomeric units of vitronectin.

“Denatured vitronectin” is a multimeric form of vitronectin formed in vitro when vitronectin is exposed to denaturing conditions; it contains more than four monomeric units of vitronectin.

In accordance with one embodiment of the invention, a sample of a biological fluid is contacted with a first antibody which binds selectively to PAI-1 in the sample to form a complex. This first antibody binds to both active and inactive PAI-1. The sample is then contacted with a second antibody which binds selectively to multimeric vitronectin. The second antibody carries a label which may be a directly detectable label or may be a component of a signal-generating system. The second antibody binds to the active PAI-1 (i.e. PAI-1/multimeric vitronectin complex)/first antibody complex. The resulting complex is separated from the reaction mixture and the second antibody bound to the complex is determined. Detection and determination of the second antibody label or the signal generated by the signal-generating system, compared with suitable calibration standards, permits measurement of the amount of PAI-1/multimeric vitronectin complex present in the sample and hence determination of active PAI-1 in the sample.

In accordance with a further embodiment, the sample is contacted with a first antibody which binds selectively to multimeric vitronectin and does not bind substantially to monomeric vitronectin. The first antibody carries a detectable label or a component of a signal-generating system. The sample is then contacted with a second antibody which binds selectively to PAI-1. Determination of the PAI-1/multimeric vitronectin complex, and of active PAI-1, is as described above.

The first and second antibodies may be added separately in a two-step procedure or may be added simultaneously.

Active PAI-1 may be determined as PAI-1/multimeric vitronectin complex by the method of the invention in a biological fluid such as whole blood, plasma, serum, urine, saliva, cerebrospinal fluid, amniotic fluid or a tissue extract.

The biological fluid is preferably whole blood, plasma or serum. When blood is collected for assay of active PAI-1 in whole blood, serum or plasma, care must be taken to avoid platelet activation, for example by using citrate as anticoagulant or by employing special blood collection tubes which promote platelet stabilisation and avoid platelet activation during blood collection; examples of suitable commercially available tubes are Stabilyte™ Blood Collection tubes, available from American Diagnostica Inc., and Becton Dickinson tubes, Catalog No. 6457.

The anti-PAI-1 antibodies used in the methods of the invention should be able to recognise PAI-1 when it is bound to multimeric vitronectin. They should therefore be directed against PAI-1 epitopes which remain exposed in the active PAI-1/vitronectin complex.

The anti-multimeric vitronectin antibodies used should recognise multimeric but not monomeric vitronectin. They should therefore be directed against epitopes exposed in multimeric vitronectin but not accessible in monomeric vitronectin. It is believed that the unique epitopes exposed in denatured vitronectin will also be present in the multimeric vitronectin of the active PAI-1/multimeric vitronectin complex. Antibodies against denatured vitronectin but which do not recognise monomeric vitronectin may therefore be used in the methods of the invention.

The antibodies used may be monoclonal or polyclonal and may be prepared by conventional techniques or obtained from commercial sources.

Anti-PAI-1 antibodies of suitable binding specificity are obtainable, for example, from American Diagnostics, Greenwich, Conn., U.S.A. (anti-PAI-1 monoclonal antibody #3780) or Biopool International, Ventura, Calif., U.S.A. (anti-PAI-1 monoclonal antibody #214101).

Anti-PAI-1 antibodies and anti-multimeric vitronectin antibodies may be prepared by conventional methods.

Either monoclonal or polyclonal antibodies with the desired binding specificity may be used in the methods of the invention. Any of the first, second or third antibodies may be a monoclonal or a polyclonal antibody. It is preferable to use monoclonal antibodies against PAI-1 and multimeric vitronectin.

Polyclonal antibodies suitable for use in the methods of the invention may be developed against PAI-1 and/or multimeric vitronectin in animals such as guinea pigs, rabbits, horses, sheep or goats, which have been immunized with purified PAI-1 or multimeric vitronectin. PAI-1 protein may be purified as described by Gils et al., (1996), Biochem., v. 35, p. 7474, or obtained commercially, for example from Molecular Innovations, Royal Oak, Mich. or American Diagnostica, Greenwich, Conn. Multimeric vitronectin may be prepared, for example, as described by Mosher et al., (1993), J. Biol. Chem., v. 268, p. 24838.

Specific protocols for the production of polyclonal antibodies are well known in the art. Briefly, the method comprises the following steps; (a) administering the selected antigen to an animal in an amount sufficient to induce the production of antibodies; (b) collecting the antisera containing said antibodies from the immunized animal; and (c) recovering the antibodies from the antisera. In order to increase the immunogenecity of the antigens, various adjuvants may be used, depending on the host species, including Freund's adjuvant (complete and incomplete), aluminum hydroxide, surface-active substances such as lysolecithin, polyanions, emulsions of oil and keyhole limpet hemocyanins.

Monoclonal anti-PAI-1 or anti-multimeric vitronectin antibodies may also be produced by methods well known in the art. Briefly, the purified protein is injected in Freund's adjuvant into mice over a suitable period of time, spleen cells are harvested and these are fused with a permanently growing myeloma partner and the resultant hybridomas are screened to identify cells producing the desired antibody with the required binding selectivity. Suitable methods for antibody preparation may be found in standard texts such as Antibody Engineering, 2d. edition, Barreback, Ed., Oxford University Press (1995).

Monoclonal antibodies produced by a selected hybridoma clone may be purified by known techniques such as ammonium sulfate fractionation, DEAE cellulose chromatography or affinity chromatography utilizing protein G or A-Sepharose column chromatography, cellulose membranes and agarose and synthetic materials such as cross-linked polysaccharides, polyvinylchloride, polypropylene, polystyrene and the like or their combinations.

Anti-PAI-1 antibodies displaying the desired binding specificity, as described above, may be obtained using screening methods similar to those described by Declerck et al., (1988), Blood, v. 71, p. 220, and anti-multimeric vitronectin antibodies may be screened for desired binding specificity as described by Sockman et al., (1993), v. 268, p. 22874 or Seiffert et al., (1994), J. Biol. Chem., v. 269, p. 2659.

The second antibody carries a label which may be any suitable directly detectable label or a component of any suitable signal-generating system. Many examples of these are well known from the field of immunoassay.

Labelling of the second antibody with a detectable label or a component of a signal-generating system may be carried out by techniques well known in the art. Examples of labels that can be utilized to render an antibody detectable include radioisotopes, enzymes, fluorescent and chemiluminescent substances. For example, a radioactive element may be used as a directly detectable label; exemplary radioactive labels include the γ-emitters ¹²⁴I, ¹²⁵I, ¹²⁸I, and ¹³¹I. A fluorescent label may also be used as a directly detectable label; for example, suitable fluorophores include coumarins such as umbelliferone, rare earth metal ions, chelates or chelate complexes, fluoresceins, rhodamine and rhodamine derivatives.

Suitable labels also include metal complexes, stable free radicals, vesicles, liposomes, colloidal particles, latex particles, spin labels, biotin/avidin and their derivatives.

Chemiluminescent labels include cyclic diacyl hydrazides, including luminol and isoluminol, acridinium esters and related compounds, pyridopyridazines, dioxeranes and bioluminescent proteins such as luciferases.

Enzyme-linked signal-generating systems may be used, including alkaline phosphatase, amylase, luciferase, catalase, beta-galactosidase, glucose oxidase, glucose-6-phosphate dehydrogenase, hexokinase, horseradish peroxidase, lactamase, urease and malate dehydrogenase. The activity of the enzyme can be detected by measuring absorbency, fluorescence or luminescence intensity after reacting the enzyme with an appropriate substrate. When enzymes are used as a label, the linkage between enzyme and antibody may be achieved by conventional methods such as glutaraldehyde, periodic acid and maleimide methods.

Solid matrices to act as solid supports suitable for immobilizing an antibody include microtitre plates, such as those obtainable from Falcon Plastics, Oxnard, Calif., or, for example, regular ELISA microtitre plates (Immulon II, Dynax, Chantilly, V.A.) and Streptavidin-coated ELISA microtitre plates (Reacti-Bind, Pierce, Rockford, Ill., and microtitre strips, such as those obtainable from Dynatech, Alexandria, Va. The wells of the strips or the microtitre plates are made of clear plastic material, preferably polyvinyl chloride or polystyrene. Other solid matrices useful for antibody immobilisation include polystyrene tubes, sticks or paddles of any convenient size, polystyrene beads, polyacrylamide matrices, paramagnetic particles, latex particles or gelatin particles.

Antibodies may be immobilised on a solid support by conventional methods which are well known in the art, for example as described in U.S. Pat. No. 5,352,583.

In accordance with a preferred embodiment of the invention, a sample of a biological fluid is contacted with a first antibody which binds selectively to PAI-1 to form a complex, the first antibody being immobilised on a solid support. Sufficient time is allowed to permit binding of the PAI-1 of the sample to the immobilised antibody. The solid support is then washed and contacted with a second antibody which binds selectively to multimeric vitronectin and is labelled with a detectable label or has attached to it a signal-generating system. The label or generated signal bound to the solid support is determined, providing a measure of the PAI-1/multimeric vitronectin complex present in the sample, and hence determining the level of active PAI-1.

In accordance with a more preferred embodiment, the sample is contacted simultaneously with the immobilised first antibody on the solid support and the labelled second antibody.

In a further embodiment, the second antibody may lack a label or signal-generating system component and the solid support-bound second antibody is determined by means of a third antibody bearing a detectable label or signal-generating system component, the third antibody binding selectively to the bound second antibody.

In accordance with a further embodiment, the sample is contacted, either simultaneously or stepwise, with a first antibody which binds selectively to PAI-1 and to which is attached one member of a capture pair and with a labelled second antibody which binds selectively to multimeric vitronectin. The resulting mixture is then contacted with a solid support on which is immobilised the other member of the capture pair. After allowing sufficient time for the labelled PAI-1/multimeric vitronectin complex to bind to the solid support by interaction of the members of the capture pair, the solid support is washed and the amount of label bound to it is determined, to determine the level of active PAI-1 in the sample. Suitable capture pairs include, for example, biotin/streptavidin. The binding selectivities of the antibodies may be reversed, the first antibody binding selectively to multimeric vitronectin and the labelled second antibody binding selectively to PAI-1.

For example, the first antibody binds selectively to PAI-1 and is biotinylated, while the second antibody, selective for multimeric vitronectin, is labelled with horse radish peroxidase (HRP). The sample/antibody mixture is placed in wells coated with streptavidin. After binding of the complex, the wells are washed and the HRP label is developed by addition of substrate and determined.

In accordance with a further embodiment, active PAI-1 may be determined in a homogeneous assay system, without separation of the PAI-1/multimeric vitronectin/first antibody/second antibody complex; such assays employ a labelled antibody wherein the label displays a detectable change on binding of the antibody, distinguishable from the label attached to unbound antibody. Examples of such assay systems, which can readily be adapted by one of ordinary skill in the art to determination of active PAI-1 by measurement of PAI-1/multimeric vitronectin complex, as described herein, are disclosed in U.S. Pat. No. 4,692,404 which employs an enzyme-labelled antibody and wherein the antibody-bound enzyme is hindered from reaction with its substrate on antigen binding of the antibody; U.S. Pat. No. 5,070,025; U.S. Pat. No. 4,318,707; U.S. Pat. No. 5,589,401 and U.S. Pat. No. 5,017,009, the contents of all of which are incorporated herein by reference.

In accordance with a further embodiment, the invention provides a kit for determining active PAI-1 in a biological fluid. The kit comprises (a) a first antibody which binds selectively to PAI-1 and (b) a labelled second antibody which binds selectively to multimeric vitronectin or a second antibody which binds selectively to multimeric vitronectin and a labelled third antibody which binds selectively to the second antibody.

In accordance with a further embodiment, the kit comprises (a) a first antibody which binds selectively to multimeric vitronectin and (b) a labelled second antibody which binds selectively to PAI-1 or a second antibody which binds selectively to PAI-1 and a labelled third antibody which binds selectively to the second antibody.

The anti-PAI-1 or anti-multimeric vitronectin first antibody may be immobilised on a solid support.

The kit may also contain a set of calibration standards. The kit may also optionally contain additional reagents such as diluents or buffers which are employed in the methods of the invention and calibration standards.

### EXAMPLES

The examples are described for the purposes of illustration and are not intended to limit the scope of the invention.

### Example 1

### Reagents

| | |
|---|---|
| Coating buffer (CB): | 40 mM K/phosphate buffer, pH 7.4 |
| | 100 mM NaCl |
| Blocking buffer (BB): | 40 mM K/phosphate buffer, pH 7.4 |
| | 100 mM NaCl |
| | 1% hydrolysed casein |
| Incubation buffer (IB): | 40 mM K/phosphate buffer, pH 7.4 |
| | 100 mM NaCl |
| | 5 mM EDTA |
| | 1% hydrolyzed casein |
| | 0.025% Tween-20 |
| Washing buffer (WB): | 40 mM K/phosphate buffer, pH 7.4 |
| | 100 mM NaCl |
| | 0.025% Tween-20 |

ELISA plates (Immulon II, Dynax)
First antibody: monoclonal anti-PAI-1 antibody
Second antibody: HRP-labelled anti-multimeric vitronectin antibody Human active recombinant PAI-1: prepared as described by Gils et al., (1996), Biochemistry, v. 35, pp. 7474-7481 or obtained commercially (American Diagnostica, Greenwich, CT or Molecular Innovations, Royal Oak, MI).

Calibration standards are prepared as follows:

In making a Vn/PAI-1 complex for the standard, an excess of Vn is utilized in order to ensure that no free active PAI-1 is left unbound. Multimeric vitronectin (mVn) at concentration of 1.3 μm is mixed with human rPAI-1 at a concentration of 0.37 μm and incubated at ambient temperature for 30 minutes. The mixture is then diluted in PAI-1 free plasma (Biopool International, Ventura, Calif. or American Diagnostica, Greenwich, Conn.) to concentration of a 200 ng of PAI-1/mL, then serially diluted in PAI-1-free plasma and stored frozen at −70C.

The wells of a regular ELISA microtitre plate (Immulon II) are coated with 100 μl/well of CB containing anti-PAI-1 monoclonal antibody (5-15 μg/ml). Plates are incubated at 4° C. for 16 to 18 hours, washed three times with WB, blocked with 200 μl/well BB for 1 hour and washed three times with WB.

50 μl portions of plasma samples or of various concentrations of PAI-1/mVn complex standards (prepared as above: final concentrations of PAI-1 in the PAI-1/mVn complex range from 0 to 100 ng/ml) are added to wells, followed by 50 μl/well HRP-labelled anti-mVn monoclonal antibody (2-5 μg/ml in IB). The plates are incubated at room temperature for 60 minutes with shaking, washed three times with WB and developed with HRP substrate for 15 minutes according to manufacturer's instructions (Sigma, St. Louis, Mo.).

The enzyme reaction is terminated by addition of 100 μl/well concentrated sulfuric acid. The intensity of the resulting colour is determined by reading the absorbency at 492 nm in a microtitre plate reader (Automated Plate Reader MR1200, Dynax, Chantilly Va.). The concentration of active PAI-1 in a sample is determined by comparison with the calibration curve.

### Example 2

Reagents are as described in Example 1. The wells of an ELISA microtitre plate are coated with 100 μL/well of CB containing anti-PAI-1 monoclonal antibody (5-15 μg/ml). The plates are incubated at 4° C. for 16-18 hours, the wells are washed three times with WB, blocked with 200 μL/well of BB for 1 hour and then washed three times with WB.

50 μL portions of the plasma samples under testing or of the various concentrations of the PAI-1-mVn complex standards (final concentration of PAI-1 in the PAI-1/mVn complex range from 0 to 100 ng/ml) are added to each well followed by 50 μL well of IB. The plates are then incubate at room temperature with shaking for 60 min. and, washed three times with WB.

100 μL of HRP-labelled anti-mVn monoclonal antibody (2-5 μg/ml) in IB is added to each well, the plates are then incubated at room temperature with shaking for 60 min, washed three times with WB and developed with the HRP substrate for 15 minutes according to the manufacturer's instructions.

The enzyme reaction is terminated by addition of 100 μL/well of concentrated sulfuric acid. The intensity of the resulting colour is determined by reading adsorbancy at 492 nm in the microtitre plate reader. The concentration of active PAI-1 in a sample is determined by comparison with the calibration curve.

### Example 3

Reagents are as described in Example 1 except for the second antibody which is biotinylated and an HRP-conjugated Streptavidin detection system is utilized, to measure bound second antibody.

The wells of an ELISA microtitre plate are coated with 100 μL/well of CB containing anti-PAI-1 monoclonal antibody (5-151 g/ml). The plates are incubated at 4° C. for 16-18 hours, the wells are washed three times with WB, blocked with 200 μL/well of BB for 1 hour and then washed three times with WB.

50 μL portions of the plasma samples under testing or of the various concentrations of the PAI-1-mVn complex standards (final concentration of PAI-1 in the PAI-1/mVn complex range from 0 to 100 μg/ml) are added to each well, followed by 50 μL/well of biotinylated anti-mVn antibody in IB, at concentration of between 2-5 g/ml. The plates are then incubated at room temperature with shaking for 60 min. and washed three times with WB.

100 μl of HRP-conjugated Streptavidin is added to each well and incubated for 30 min at room temperature with shaking. The plate is washed three times with WB and then developed with the HRP substrate for 15 minutes according to the manufacturer's instructions.

The enzyme reaction is terminated by addition of 100 μL/well of concentrated sulfuric acid. The intensity of the resulting colour is determined by reading adsorbancy at 492 nm in the microtitre plate reader. The concentration of active PAI-1 in a sample is determined by comparison with the calibration curve.

### Example 4

Reagents are as described in Example 1 except that the anti-PAI-1 first antibody is conjugated with biotin and the anti-mVn second antibody is labelled with HRP.

Test tubes are used for performing the immune complex formation and then the immune complex binding and development are performed in the wells of streptavidin-coated ELISA microtitre plates (Reacti-Bind, Pierce, Rockford Ill.).

### Procedure:

50 μL of biotinylated anti-PAI-1 antibody (10-15 μg/ml) in IB is added to a test tube, followed by 50 μL of HRP labelled anti-mVn antibody (5-151 g/ml) in IB, and then 100 μL of sample to be tested or of the various concentrations of the PAI-1-mVn complex standards (final concentration of PAI-1 in the PAI-1/mVn complex range from 0 to 100 ng/ml). Test tubes are incubated for 60 minutes at room temperature with shaking. Simultaneously, the wells of a Streptavidin-coated microtitre plate are blocked with 200 μL of BB and washed three times with WB.

100 μL of reaction mixture is transferred from each test tube to a well of the blocked Streptavidin-coated microtitre plate and the plate is incubated for 30 minutes at room temperature with shaking. The plate is washed three times with WB and then developed with the HRP substrate for 15 minutes according to the manufacturer's instructions.

The enzyme reaction is terminated by addition of 100 μL/well of concentrated sulfuric acid. The intensity of the resulting colour is determined by reading adsorbancy at 492 nm in the microtitre plate reader. The concentration of active PAI-1 in the sample is determined by comparison with the calibration curve.

The present invention is not limited to the features of the embodiments described herein, but includes all variations and modifications within the scope of the claims.

1. (canceled)

2. A method for determining active plasminogen activator inhibitor-Type 1 (PAI-1) in a biological fluid, the method comprising the steps of:
(i) providing a sample of a biological fluid selected from the group consisting of whole blood, plasma and serum;
(ii) measuring the amount of active PAI-1/multimeric vitronectin complex in the sample; and
(iii) determining the amount of active PAI-1 in the biological fluid by correlating the amount of active PAI-1 in the biological fluid to the amount of active PAI-1/multimeric vitronectin complex in the sample, wherein measuring the amount of active PAI-1/multimeric vitronectin complex in the sample comprises:
(a) contacting the sample either simultaneously or stepwise with a first antibody which binds selectively to PAI-1 and a labelled second antibody which binds selectively to multimeric vitronectin; and
(b) determining the second antibody bound to the complex to measure the amount of active PAI-1/multimeric vitronectin complex in the sample.

3. A method for determining active plasminogen activator inhibitor-Type 1 (PAI-1) in a biological fluid, the method comprising the steps of:
(i) providing a sample of a biological fluid selected from the group consisting of whole blood, plasma and serum;
(ii) measuring the amount of active PAI-1/multimeric vitronectin complex in the sample; and
(iii) determining the amount of active PAI-1 in the biological fluid by correlating the amount of active PAI-1 in the biological fluid to the amount of active PAT-1/multimeric vitronectin complex in the sample, wherein measuring the amount of active PAI-1/multimeric vitronectin complex in the sample comprises:
(a) contacting the sample either simultaneously or stepwise with a first antibody which binds selectively to multimeric vitronectin and a labelled second antibody which binds selectively to PAI-1; and
(b) determining the second antibody bound to the complex to measure the amount of active PAI-1/multimeric vitronectin complex in the sample.

4. A method for determining active plasminogen activator inhibitor-Type 1 (PAI-1) in a biological fluid, the method comprising the steps of:
(i) providing a sample of a biological fluid selected from the group consisting of whole blood, plasma and serum;
(ii) measuring the amount of active PAI-1/multimeric vitronectin complex in the sample; and
(iii) determining the amount of active PAI-1 in the biological fluid by correlating the amount of active PAI-1 in the biological fluid to the amount of active PAI-1/multimeric vitronectin complex in the sample, wherein measuring the amount of active PAI-1/multimeric vitronectin complex in the sample comprises:
(a) contacting the sample either simultaneously or stepwise with a first antibody which binds selectively to PAI-1 and a labelled second antibody which binds selectively to multimeric vitronectin to form an active PAI-1/multimeric vitronectin/first antibody/second antibody complex;
(b) separating said active PAI-1/multimeric vitronectin/first antibody/second antibody complex from the sample; and
(c) determining the second antibody bound to the complex to measure the amount of active PAI-1/multimeric vitronectin complex in the sample.

5. A method for determining active plasminogen activator inhibitor-Type 1 (PAI-1) in a biological fluid, the method comprising the steps of:
(i) providing a sample of a biological fluid selected from the group consisting of whole blood, plasma and serum;
(ii) measuring the amount of active PAI-1/multimeric vitronectin complex in the sample; and
(iii) determining the amount of active PAI-1 in the biological fluid by correlating the amount of active PAI-1 in the biological fluid to the amount of active PAI-1/multimeric vitronectin complex in the sample, wherein measuring the amount of active PAI-1/multimeric vitronectin complex in the sample comprises:
a) contacting the sample either simultaneously or stepwise with a first antibody which binds selectively to multimeric vitronectin and a labelled second antibody which binds selectively to PAI-1 to form an active PAI-1/multimeric vitronectin/first antibody/second antibody complex;
(b) separating said active PAI-1/multimeric vitronectin/first antibody/second antibody complex from the sample; and
(c) determining the second antibody bound to the complex to measure the amount of active PAI-1/multimeric vitronectin complex in the sample.

6. A method for determining active plasminogen activator inhibitor-Type 1 (PAI-1) in a biological fluid, the method comprising the steps of:
(i) providing a sample of a biological fluid selected from the group consisting of whole blood, plasma and serum;
(ii) measuring the amount of active PAI-1/multimeric vitronectin complex in the sample; and
(iii) determining the amount of active PAI-1 in the biological fluid by correlating the amount of active PAI-1 in the biological fluid to the amount of active PAI-1/multimeric vitronectin complex in the sample, wherein measuring the amount of active PAI-1/multimeric vitronectin complex in the sample comprises:
(a) simultaneously contacting the sample with a first antibody which binds selectively to PAI-1, the first antibody being immobilized on a solid support, and with a labelled second antibody which binds selectively to multimeric vitronectin; and
(b) determining the second antibody bound to the solid support to measure the amount of active PAI-1/multimeric vitronectin complex in the sample.

7. A method for determining active plasminogen activator inhibitor-Type 1 (PAI-1) in a biological fluid, the method comprising the steps of:
(i) providing a sample of a biological fluid selected from the group consisting of whole blood, plasma and serum;
(ii) measuring the amount of active PAI-1/multimeric vitronectin complex in the sample; and
(iii) determining the amount of active PAI-1 in the biological fluid by correlating the amount of active PAI-1 in the biological fluid to the amount of active PAI-1/multimeric vitronectin complex in the sample, wherein measuring the amount of active PAI-1/multimeric vitronectin complex in the sample comprises:
(a) contacting the sample with a first antibody which binds selectively to PAI-1, the first antibody being immobilized on a solid support;
(b) contacting the solid support with a labelled second antibody which binds selectively to multimeric vitronectin; and
(c) determining the second antibody bound to the solid support to measure the amount of active PAI-1/multimeric vitronectin complex in the sample.

8. A method for determining active plasminogen activator inhibitor-Type 1 (PAI-1) in a biological fluid, the method comprising the steps of:
(i) providing a sample of a biological fluid selected from the group consisting of whole blood, plasma and serum;
(ii) measuring the amount of active PAI-1/multimeric vitronectin complex in the sample; and
(iii) determining the amount of active PAI-1 in the biological fluid by correlating the amount of active PAI-1 in the biological fluid to the amount of active PAI-1/multimeric vitronectin complex in the sample, wherein measuring the amount of active PAI-1/multimeric vitronectin complex in the sample comprises:
(a) simultaneously contacting the sample with a first antibody which binds selectively to multimeric vitronectin, the first antibody being immobilized on a solid support, and with a labelled second antibody which binds selectively to PAI-1; and
(b) determining the second antibody bound to the solid support to measure the amount of active PAI-1/multimeric vitronectin complex in the sample.

9. A method for determining active plasminogen activator inhibitor-Type 1 (PAI-1) in a biological fluid, the method comprising the steps of:
(i) providing a sample of a biological fluid selected from the group consisting of whole blood, plasma and serum;
(ii) measuring the amount of active PAI-1/multimeric vitronectin complex in the sample; and
(iii) determining the amount of active PAI-1 in the biological fluid by correlating the amount of active PAI-1 in the biological fluid to the amount of active PAI-1/multimeric vitronectin complex in the sample, wherein measuring the amount of active PAI-1/multimeric vitronectin complex in the sample comprises:
(a) contacting the sample with a first antibody which binds selectively to multimeric vitronectin, the first antibody being immobilized on a solid support;
(b) contacting the solid support with a labelled second antibody which binds selectively to PAI-1; and
(c) determining the second antibody bound to the solid support to measure the amount of active PAI-1/multimeric vitronectin complex in the sample.

10. A method for determining active plasminogen activator inhibitor-Type 1 (PAI-1) in a biological fluid, the method comprising the steps of:
(i) providing a sample of a biological fluid selected from the group consisting of whole blood, plasma and serum;
(ii) measuring the amount of active PAI-1/multimeric vitronectin complex in the sample; and
(iii) determining the amount of active PAI-1 in the biological fluid by correlating the amount of active PAI-1 in the biological fluid to the amount of active PAT-1/multimeric vitronectin complex in the sample, wherein measuring the amount of active PAI-1/multimeric vitronectin complex in the sample comprises:
(a) contacting the sample with a first antibody which binds selectively to PAI-1, the first antibody being immobilized on a solid support;
(b) contacting the solid support with a second antibody which binds selectively to multimeric vitronectin;
(c) contacting the solid support with a labelled third antibody which binds selectively to the second antibody; and
(d) determining the third antibody bound to the solid support to measure the amount of active PAI-1/multimeric vitronectin complex in the sample.

11. A method for determining active plasminogen activator inhibitor-Type 1 (PAI-1) in a biological fluid, the method comprising the steps of:
(i) providing a sample of a biological fluid selected from the group consisting of whole blood, plasma and serum;
(ii) measuring the amount of active PAI-1/multimeric vitronectin complex in the sample; and
(iii) determining the amount of active PAI-1 in the biological fluid by correlating the amount of active PAI-1 in the biological fluid to the amount of active PAI-1/multimeric vitronectin complex in the sample, wherein measuring the amount of active PAI-1/multimeric vitronectin complex in the sample comprises:
(a) contacting the sample with a first antibody which binds selectively to multimeric vitronectin, the first antibody being immobilized on a solid support;
(b) contacting the solid support with a second antibody which binds selectively to PAI-1;
(c) contacting the solid support with a labelled third antibody which binds selectively to the second antibody; and
(d) determining the third antibody bound to the solid support to measure the amount of active PAI-1/multimeric vitronectin complex in the sample.

12. A method for determining active plasminogen activator inhibitor-Type 1 (PAI-1) in a biological fluid, the method comprising the steps of:
(i) providing a sample of a biological fluid selected from the group consisting of whole blood, plasma and serum;
(ii) measuring the amount of active PAI-1/multimeric vitronectin complex in the sample; and
(iii) determining the amount of active PAI-1 in the biological fluid by correlating the amount of active PAI-1 in the biological fluid to the amount of active PAI-1/multimeric vitronectin complex in the sample, wherein measuring the amount of active PAI-1/multimeric vitronectin complex in the sample comprises:
(a) contacting the sample, either simultaneously or stepwise, with a first antibody which binds selectively to PAI-1 and to which is attached one member of a capture pair and with a labelled second antibody which binds selectively to multimeric vitronectin to form a mixture;
(b) contacting the mixture with a solid support on which is immobilized the other member of the capture pair; and
(c) determining the second antibody bound to the solid support to measure the amount of active PAI-1/multimeric vitronectin complex in the sample.

13. A method for determining active plasminogen activator inhibitor-Type 1 (PAI-1) in a biological fluid, the method comprising the steps of:
(i) providing a sample of a biological fluid selected from the group consisting of whole blood, plasma and serum;
(ii) measuring the amount of active PAI-1/multimeric vitronectin complex in the sample; and
(iii) determining the amount of active PAT-1 in the biological fluid by correlating the amount of active PAI-1 in the biological fluid to the amount of active PAI-1/multimeric vitronectin complex in the sample, wherein measuring the amount of active PAI-1/multimeric vitronectin complex in the sample comprises:
(a) contacting the sample either simultaneously or stepwise, with a first antibody which binds selectively to multimeric vitronectin and to which is attached one member of a capture pair and with a labelled second antibody which binds selectively to PAI-1 to form a mixture;
(b) contacting the mixture with a solid support on which is immobilized the other member of the capture pair; and
(c) determining the second antibody bound to the solid support to measure the amount of active PAI-1/multimeric vitronectin complex in the sample.

14-15. (canceled)

16. The method according to claim 3, wherein the second antibody is labelled with a directly detectable label.

17. The method according to claim 3, wherein the second antibody is labelled with a component of a signal-generating system.

18. The method of claim 17 wherein the component is an enzyme selected from the group consisting of alkaline phosphatase, amylase, luciferase, catalase, beta-galactosidase, glucose oxidase, glucose-6-phosphate dehydrogenase, hexokinase, horseradish peroxidase, lactamase, urease and malate dehydrogenase.

19. The method according to claim 3, wherein the second antibody is labelled with a fluorophore.

20. The method of claim 19 wherein the fluorophore is selected from the group consisting of a coumarin, a rare earth metal ion, chelate or chelate complex, a fluorescein, rhodamine and a rhodamine derivative.

21. The method of claim 3, wherein the second antibody is labelled with a luminescent material.

22. The method of claim 21 wherein the luminescent material is selected from the group consisting of a cyclic diacyl hydrazide, luminol, isoluminol, an acridinium ester, a pyridopyridazine, a dioxerane, a bioluminescent protein and a luciferase.

23. The method of claim 3 wherein the second antibody is labelled with a label selected from the group consisting of a metal complex, a stable free radical, a vesicle, a liposome, a colloidal particle, a latex particle, a spin label, biotin and avidin.

24. The method of claim 6, wherein the solid support is selected from the group consisting of an ELISA plate, a polyacrylamide matrix, a polystyrene tube, polystyrene beads, latex particles, paramagnetic particles, acrylic particles and gelatin particles.

25-31. (canceled)
